(19) United States
(12) Patent Application Publication
(10) Pub. No.: US 2025/0200162 A1
(43) Pub. Date: Jun. 19, 2025

(54) INFORMATION PROCESSING APPARATUS AND METHOD, STORAGE MEDIUM, AND AUTHENTICATION APPARATUS

(71) Applicant: JVCKENWOOD Corporation, Yokohama-shi (JP)

(72) Inventor: Shohei Odan, Yokohama-shi (JP)

(21) Appl. No.: 19/073,108

(22) Filed: Mar. 7, 2025

Related U.S. Application Data
(63) Continuation of application No. PCT/JP2023/032682, filed on Sep. 7, 2023.

(30) Foreign Application Priority Data
Sep. 15, 2022 (JP) 2022-147286

(51) Int. Cl. G06F 21/32

(52) U.S. Cl. CPC G06F 21/32

(57) ABSTRACT

A stimulation unit that applies a plurality of stimulations having first relationship, a biological information acquisition unit that acquires a brain wave level for the plurality of stimulations applied by the stimulation unit, and a personal information setting unit that sets, as a personal information determination value, first personal information associating the plurality of stimulations having the first relationship that has been applied by the stimulation unit, and a difference value of a plurality of brain wave levels acquired by the biological information acquisition unit at this time are included.

### CROSS-REFERENCE TO RELATED APPLICATIONS

This application is a Continuation of PCT International Application No. PCT/JP2023/032682 filed on Sep. 7, 2023 which claims the benefit of priority from Japanese Patent Application No. 2022-147286 filed on Sep. 15, 2022, the entire contents of both of which are incorporated herein by reference.

### BACKGROUND OF THE INVENTION

### 1. Field of the Invention

The present disclosure relates to an information processing apparatus and a method, a program, and an authentication apparatus.

### 2. Description of the Related Art

Personal authentication methods include a method that uses a password, but if a password is identified, an unauthorized access by impersonation becomes possible. Further, as another authentication method, there is biometric authentication that uses a fingerprint, a face, or the like, but the biometric authentication also has limitations, and high confidentiality such as privacy protection needs to be ensured. On the other hand, for example, JP 2015-84970 A described below describes a technique of predicting a personal characteristic from a captured image of a brain of a user.

The technique described in JP 2015-84970 A detects a characteristic value of each region of a brain based on three-dimensional information of a brain cortex that is obtained from a brain image of the user, and predicts the ability and qualification of the user by comparing the characteristic value with stored information prepared in advance. Meanwhile, in this case, only by simply acquiring the three-dimensional information of the brain cortex from the brain image of the user, it is difficult to acquire information unique to the user.

### SUMMARY OF THE INVENTION

It is an object of the present invention to at least partially solve the problems in the conventional technology.

The above and other objects, features, advantages and technical and industrial significance of this invention will be better understood by reading the following detailed description of presently preferred embodiments of the invention, when considered in connection with the accompanying drawings.

An information processing apparatus according to a present embodiment comprising: a stimulation unit configured to apply a plurality of stimulations having first relationship; a biological information acquisition unit configured to acquire a brain wave level for the plurality of stimulations applied by the stimulation unit; and a personal information setting unit configured to set, as a personal information determination value, first personal information associating the plurality of stimulations having the first relationship that has been applied by the stimulation unit, and a difference value of a plurality of brain wave levels acquired by the biological information acquisition unit at this time.

An information processing method according to a present embodiment comprising: a step of applying a plurality of stimulations having first relationship;
a step of acquiring a brain wave level for the plurality of applied stimulations; and a step of setting, as a personal information determination value, first personal information associating the plurality of applied stimulations having the first relationship, and a difference value of a plurality of brain wave levels acquired at this time.

A non-transitory computer-readable storage medium storing a program according to a present embodiment for causing a computer operating as an information processing apparatus, to execute: a step of applying a plurality of stimulations having first relationship; a step of acquiring a brain wave level for the plurality of applied stimulations; and a step of setting, as a personal information determination value, first personal information associating the plurality of applied stimulations having the first relationship, and a difference value of a plurality of brain wave levels acquired at this time.

### BRIEF DESCRIPTION OF THE DRAWINGS

FIG. 1 is a block diagram illustrating an information processing apparatus according to a first embodiment;

FIG. 2 is a schematic diagram for describing a specific example of a stimulation pattern to be presented;

FIG. 3 is a schematic diagram for describing a detected value for a presented stimulation pattern;

FIG. 4 is a flowchart illustrating an information processing method according to the first embodiment;

FIG. 5 is a block diagram illustrating an authentication apparatus according to a second embodiment; and

FIG. 6 is a flowchart illustrating an authentication method according to the second embodiment.

### DETAILED DESCRIPTION OF THE PREFERRED EMBODIMENTS

Hereinafter, embodiments of an information processing apparatus and a method, a program, and an authentication apparatus according to the present disclosure will be described in detail with reference to the accompanying drawings. Note that the present invention is not limited by the following embodiment.

### First Embodiment

### Information Processing Apparatus

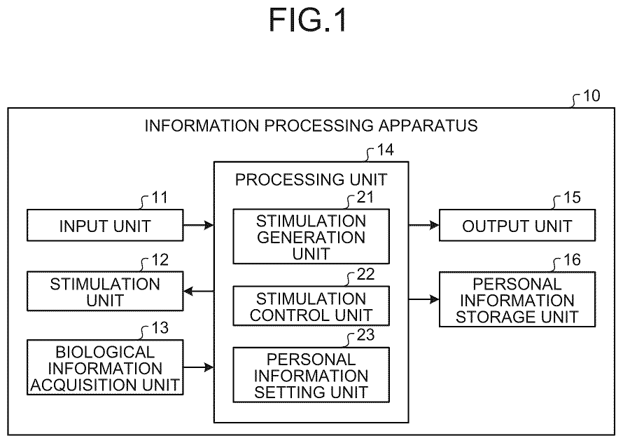

FIG. 1 is a block diagram illustrating an information processing apparatus according to the present embodiment.

As illustrated in FIG. 1, an information processing apparatus 10 includes an input unit 11, a stimulation unit 12, a biological information acquisition unit 13, a processing unit 14, an output unit 15, and a personal information storage unit 16.

The input unit 11 is connected to the processing unit 14. The input unit 11 is operable by the user, and can input various signals to the processing unit 14. The input unit 11 inputs, for example, a start signal for the user starting processing of a personal information determination value of himself/herself, and an end signal for ending the processing of the personal information determination value of the user, to the processing unit 14. The input unit 11 can be implemented by, for example, a touch panel, a button, a switch, a keyboard, or the like.

The stimulation unit 12 is connected to the processing unit 14. The stimulation unit 12 can apply a plurality of stimulations to the user. The processing unit 14 outputs a stimulation application signal to the stimulation unit 12 based on a program. The stimulation unit 12 applies a stimulation to the user based on the stimulation application signal input from the processing unit 14. The stimulation unit 12 applies a plurality of different stimulations to a predetermined number of sensory organs among five senses. The stimulation unit 12 applies a plurality of stimulations to the user at predetermined intervals.

The types of stimulations to the user include, for example, a visual sense, an auditory sense, a tactile sense, an olfactory sense, and a taste sense that are included in the five senses. The stimulation to be felt by the visual sense is a stimulation of showing an image (video) to the user. The stimulation to be felt by the auditory sense is a stimulation of causing the user to hear sound. The stimulation to be felt by the tactile sense is a stimulation of an object being touched by the user. The stimulation to be felt by the olfactory sense is a stimulation of causing the user to smell scent (aroma). The stimulation to be felt by the taste sense is a stimulation of causing the user to eat food.

Specifically, the stimulation unit 12 is a device that applies, to the user, stimulations of the visual sense, the auditory sense, the tactile sense, the olfactory sense, and the taste sense that are included in the five senses. The stimulation to be felt by the visual sense is applied by a display device such as a display, for example, the stimulation to be felt by the auditory sense is applied by an acoustic device such as a speaker or a headphone, for example, the stimulation to be felt by the tactile sense is applied by a vibrator or a heating device, and the stimulations to be felt by the olfactory sense and the taste sense are applied by a device that emits specific scent, aroma, or the like from a dedicated cartridge, or the like.

The biological information acquisition unit 13 is connected to the processing unit 14. The biological information acquisition unit 13 acquires biological information obtainable when the stimulation unit 12 applies a stimulation to the user. That is, if the processing unit 14 outputs a stimulation application signal to the stimulation unit 12 based on a program, the stimulation unit 12 applies a stimulation to the user. At this time, the processing unit 14 outputs a biological information acquisition signal to the biological information acquisition unit 13 based on a program. Accordingly, the biological information acquisition unit 13 acquires biological information of the user based on the biological information acquisition signal input from the processing unit 14.

The biological information acquisition unit 13 measures, as biological information, a brain wave level, a brain wave measurement position, and a pulse wave level. The biological information acquisition unit 13 acquires brain waves being brain information of the user, for example. The biological information acquisition unit 13 has an electric sensor (for example, electrode) that detects brain waves output from weak current flowing in a cranial nerve network, for example. When the user receives a stimulation from the outside, the biological information acquisition unit 13 detects an electric potential (electric signal) of weak current that is based on a thought such as thinking of the user. Note that the biological information acquisition unit 13 is not limited to the electric sensor. Further, the pulse wave level is measured by a transmissive photoelectric pulse sensor including a light emission unit and a light receiving unit, for example.

The processing unit 14 includes a stimulation generation unit 21, a stimulation control unit 22, and a personal information setting unit 23. Note that the processing unit 14 is a control device, and includes an arithmetic circuit such as a central processing unit (CPU), for example. Further, a storage unit (not illustrated) storing programs for performing various types of processing is connected to the processing unit 14.

The stimulation generation unit 21 generates a pattern by which the stimulation unit 12 applies a plurality of stimulations to the user. The stimulation generation unit 21 generates a plurality of stimulation patterns. A stimulation pattern includes a plurality of stimulations having relationship. A specific example of the stimulation pattern will be described later.

The stimulation control unit 22 outputs a stimulation application signal of one stimulation pattern or a plurality of stimulation patterns generated by the stimulation generation unit 21, to the stimulation unit 12 based on a program. Further, simultaneously with outputting a stimulation application signal to the stimulation unit 12, the stimulation control unit 22 outputs a biological acquisition signal corresponding to the stimulation pattern, to the biological information acquisition unit 13 based on a program.

The stimulation unit 12 accordingly applies a stimulation to the user based on the stimulation application signal input from the stimulation control unit 22. Then, the biological information acquisition unit 13 acquires biological information of the user based on a biological information acquisition signal input from the stimulation control unit 22.

The personal information setting unit 23 sets, as a personal information determination value, first personal information associating a stimulation pattern including a plurality of stimulations having relationship that has been applied to the user by the stimulation unit 12, and a plurality of pieces of biological information acquired by the biological information acquisition unit 13 at this time (specifically, a difference value of a plurality of brain wave levels). The personal information determination value is for authenticating the user, and authentication of the user is performed by comparing the personal information determination value with personal information of the user.

The output unit 15 is connected to the processing unit 14. The output unit 15 can output a processing result of the processing unit 14 such as, for example, whether or not the stimulation unit 12 has applied a stimulation to the user, or whether or not the biological information acquisition unit 13 has acquired biological information of the user. The output unit 15 is, for example, a display device that displays an image, a video, and the like, and a sound output device that outputs sound. Note that, when the stimulation unit 12 is a display device that applies a stimulation to be felt by the visual sense, to the user, the stimulation unit 12 may also function as the display device serving as the output unit 15.

The personal information storage unit 16 is connected to the processing unit 14. The personal information storage unit 16 stores various types of information. The personal information storage unit 16 stores a personal information determination value set by the personal information setting unit 23. At this time, the personal information storage unit 16 stores the acquired personal information determination value of the user in association with an acquisition date, an ID (personal identification number) of the user, or the like. Note that the personal information storage unit 16 is an external storage device such as a hard disk drive (HDD), a memory, or the like.

### Specific Example of Stimulation Application

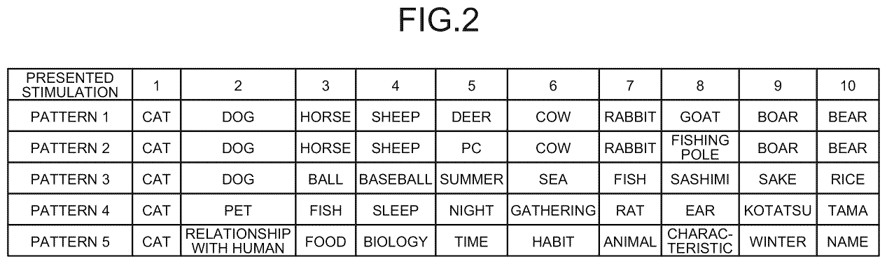

FIG. 2 is a schematic diagram for describing a specific example of a stimulation pattern to be presented.

As illustrated in FIGS. 1 and 2, the stimulation unit 12 applies a plurality of different stimulations to the user at predetermined intervals. In this case, the stimulation unit 12 applies a stimulation to the user using a stimulation pattern including a plurality of different stimulations generated by the stimulation generation unit 21. Hereinafter, a stimulation pattern will be described. Here, the visual sense is applied as the type of a stimulation, and a display device is used as a device that applies the stimulation to be felt by the visual sense, to the user. That is, the stimulation unit 12 applies a stimulation to the user by displaying a predetermined image on the display device at predetermined intervals.

In a case where the display device is used as the stimulation unit 12, examples of stimulation patterns to be presented to the user include five stimulation patterns to be described below, but the stimulation patterns are not limited to these stimulation patterns. A stimulation pattern 1 includes a plurality of stimulations having first relationship. The plurality of stimulations having the first relationship refers to a state that all the stimulations have common relationship. For example, the plurality of stimulations having the first relationship refers to stimulations having relationship of “animal”. Thus, the stimulation pattern 1 is a stimulation of displaying images of a cat, a dog, a horse, a sheep, a deer, and the like that have the relationship of “animal”. Note that the number of a plurality of stimulations in the stimulation pattern 1 is not limited. Further, aside from “animal”, the plurality of stimulations having the first relationship may be stimulations having relationship of “plant”, “home electronics”, “food”, “nature”, or the like, for example.

A stimulation pattern 2 includes a combination of a plurality of stimulations having the first relationship and stimulations not having the first relationship. When the plurality of stimulations having the first relationship corresponds to stimulations having relationship of “animal”, for example, the stimulations not having the first relationship include a personal computer (PC), a fishing pole, and the like that are unrelated to “animal”. Thus, the stimulation pattern 2 is a stimulation of displaying images of the PC, the fishing pole, and the like that do not have relationship “animal”, in the middle of the images of the cat, the dog, the horse, the sheep, the deer, and the like that have relationship of “animal”.

A stimulation pattern 3 includes a plurality of stimulations having second relationship. The plurality of stimulations having the second relationship refers to a state in which adjacent stimulations have common relationship. For example, the stimulation pattern 3 is a stimulation of displaying images of a cat, a dog, a ball, baseball, summer, sea, and the like. Because adjacent stimulations in the stimulation pattern 3 have common relationship, the cat and the dog, the dog and the ball, the ball and the baseball, the baseball and the summer, and the summer and the sea have common relationship.

A stimulation pattern 4 includes a plurality of stimulations having third relationship. The plurality of stimulations having the third relationship refers to a state in which a first stimulation and second and later stimulations have common relationship. For example, the stimulation pattern 4 is a stimulation of displaying images of a cat, a pet, a fish, sleep, night, gathering and the like. Because a first stimulation and second and later stimulations in the stimulation pattern 4 have common relationship, the cat and the pet, the cat and the fish, the cat and the sleep, the cat and the night, and the cat and the gathering have common relationship.

A stimulation pattern 5 includes a plurality of stimulations having fourth relationship. The plurality of stimulations having the fourth relationship refers to representing relationship with a first stimulation. For example, the stimulation pattern 5 is a stimulation of displaying images of a cat, relationship with human, food, biology, time, habit, or the like. Because a first stimulation and second and later stimulations in the stimulation pattern 5 have relationship, the cat and the relationship with human, the cat and the food, the cat and the biology, the cat and the time, and the cat and the habit have common relationship.

### Specific Example of Biological Acquisition

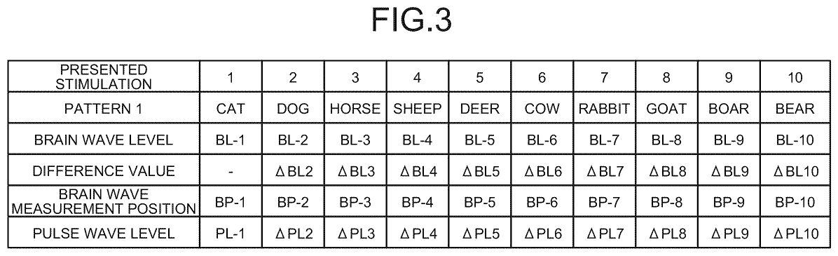

FIG. 3 is a schematic diagram for describing a detected value for a presented stimulation pattern.

As illustrated in FIGS. 1 and 3, when the stimulation unit 12 applies a plurality of different stimulations to the user at predetermined intervals, the biological information acquisition unit 13 acquires biological information of the user for each stimulation. Here, because the stimulation unit 12 applies stimulations to the user by displaying a predetermined image on a display device at predetermined intervals, the biological information acquisition unit 13 acquires biological information of the user each time the predetermined image is displayed on the display device. For example, when the stimulation unit 12 sequentially displays the images of the cat, the dog, the horse, the sheep, the deer, and the like on the display device based on the stimulation pattern 1, the biological information acquisition unit 13 sequentially acquires biological information of the user that is obtained in response to the image of the cat, biological information of the user that is obtained in response to the image of the dog, biological information of the user that is obtained in response to the image of the horse, biological information of the user that is obtained in response to the image of the sheet, biological information of the user that is obtained in response to the image of the deer, and the like.

The biological information of the user that is to be acquired by the biological information acquisition unit 13 includes a brain wave level, a brain wave measurement position, and a pulse wave level. That is, if the stimulation unit 12 displays the images of the cat, the dog, the horse, the sheep, the deer, and the like based on the stimulation pattern 1, the biological information acquisition unit 13 acquires brain wave levels BL-1, BL-2, BL-3, BL-4, BL-5, and so on of the user, brain wave measurement positions BP-1, BP-2, BP-3, BP-4, BP-5, and so on, and pulse wave levels PL-1, PL-2, PL-3, PL-4, PL-5, and so on that are obtained in response to the images of the cat, the dog, the horse, the sheep, the deer, and the like. At this time, the brain wave level is a value at the brain wave measurement position.

### Specific Example of Personal Information Setting

The personal information setting unit 23 calculates a difference value of brain wave levels based on a plurality of brain wave levels acquired by the biological information acquisition unit 13. Here, a difference value of brain wave levels is a difference value of adjacent brain wave levels among a plurality of brain wave levels acquired by the biological information acquisition unit 13. That is, the difference values of brain wave levels include a difference value ΔBL2 of the brain wave level BL-1 and the brain wave level BL-2, a difference value ΔBL3 of the brain wave level BL-2 and the brain wave level BL-3, a difference value ΔBL4 of the brain wave level BL-3 and the brain wave level BL-4, and a difference value ΔBL5 of the brain wave level BL-4 and the brain wave level BL-5. The difference value of brain wave levels in this case is preferably a relative value instead of an absolute value.

Note that the difference value of brain wave levels is not limited to a difference value of adjacent brain wave levels, and may be a difference value of a first brain wave level (reference value) and a second or later brain wave level, for example. That is, the difference values of brain wave levels include the difference value ΔBL2 of the brain wave level BL-1 and the brain wave level BL-2, the difference value ΔBL3 of the brain wave level BL-1 and the brain wave level BL-3, the difference value ΔBL4 of the brain wave level BL-1 and the brain wave level BL-4, and the difference value ΔBL5 of the brain wave level BL-1 and the brain wave level BL-5. In this case, the difference value of brain wave levels is not limited to a difference value ΔBL of brain wave levels BL being actual measured values, and the difference value ΔBL may be replaced with a difference value level (for example, level 0-10) or the like. The level 0 indicates no change and the level 10 indicates the largest change.

The personal information setting unit 23 sets, as a personal information determination value, personal information associating a stimulation pattern applied to the user by the stimulation unit 12, and a difference value of a plurality of brain wave levels acquired by the biological information acquisition unit 13 for the stimulation pattern. The personal information storage unit 16 stores the personal information determination value set by the personal information setting unit 23, in association with an acquisition date, an ID of the user, or the like.

In this case, the stimulation unit 12 applies a stimulation of one stimulation pattern to the user, and the biological information acquisition unit 13 acquires a plurality of brain wave levels for the one stimulation pattern. In other words, the stimulation unit 12 applies a plurality of stimulations having the first relationship, and the biological information acquisition unit 13 acquires brain wave levels for the plurality of stimulations having the first relationship. Thus, the personal information setting unit 23 sets, as a personal information determination value, first personal information associating a plurality of stimulations having the first relationship and a difference value of a plurality of brain wave levels acquired at this time.

Note that a plurality of stimulations having the first relationship is not limited to the above-described stimulation pattern 1, and is any one of the five stimulation patterns 1 to 5. That is, a plurality of stimulations having the first relationship includes a plurality of stimulations having the second to fourth relationships.

Nevertheless, a setting method of the personal information determination value that is used by the personal information setting unit 23 is not limited to this method. For example, the stimulation unit 12 applies stimulations of a plurality of stimulation patterns to the user, and the biological information acquisition unit 13 acquires a plurality of brain wave levels for the plurality of stimulation patterns. In other words, the stimulation unit 12 applies a plurality of stimulations having the first relationship and the second relationship, and the biological information acquisition unit 13 acquires brain wave levels for the plurality of stimulations having the first relationship and the second relationship. Thus, the personal information setting unit 23 sets the first personal information associating the plurality of stimulations having the first relationship and a difference value of a plurality of brain wave levels acquired at this time, and second personal information associating the plurality of stimulations having the second relationship and a difference value of a plurality of brain wave levels acquired at this time. Then, the personal information setting unit 23 sets the first personal information and the first personal information as personal information determination values.

Note that the plurality of stimulations having the first relationship and the second relationship is not limited to the above-described stimulation patterns 1 and 3, and is any two or more of the five stimulation patterns 1 to 5. That is, the personal information setting unit 23 may set a plurality of pieces of personal information as the personal information determination value. In this case, a simulation is any of the plurality of stimulation patterns 1 to 5.

Further, the biological information acquisition unit 13 acquires not only a brain wave level of the user, but also a brain wave measurement position at which the brain wave level has been measured, and a pulse wave level. Here, the brain wave measurement position is an upper position, a lower position, a right position, a left position, a rear position, or a front position of a brain of the user, or no change, and a difference value is preferably used as a pulse wave level. Thus, the personal information setting unit 23 may set, as the personal information determination value, personal information associating a plurality of stimulations, a difference value of a plurality of brain wave levels acquired at this time, a brain wave measurement position, and a difference value of pulse wave levels.

Note that, in the above description, a stimulation to be applied to the user by the stimulation unit 12 is assumed to be a stimulation to be felt by the visual sense, and a predetermined image is displayed on a display device. In this case, an image of an object may be displayed, or a name of an object may be displayed. Further, a stimulation to be applied to the user by the stimulation unit 12 is not limited to a stimulation to be felt by the visual sense, and may be a stimulation to be felt by the auditory sense, the tactile sense, the olfactory sense, the taste sense, or the like, or may be a stimulation to be felt by different five senses in combination.

### Information Processing Method

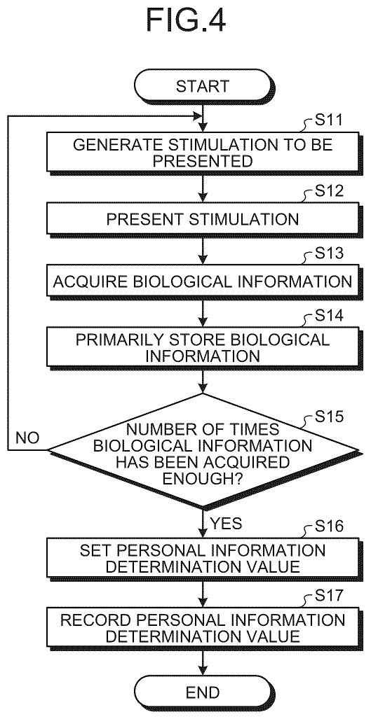

FIG. 4 is a flowchart illustrating an information processing method according to the first embodiment.

As illustrated in FIGS. 1 and 4, in Step S11, the stimulation generation unit 21 generates a pattern (stimulation pattern) by which the stimulation unit 12 applies a plurality of stimulations to the user. Note that the stimulation generation unit 21 may preliminarily generate a stimulation pattern including a plurality of stimulations. In Step S12, the stimulation control unit 22 outputs a stimulation application signal of the stimulation pattern generated by the stimulation generation unit 21, to the stimulation unit 12. The stimulation unit 12 accordingly applies a stimulation to the user based on the stimulation application signal input from the stimulation control unit 22.

In Step S13, simultaneously with outputting the stimulation application signal to the stimulation unit 12, the stimulation control unit 22 outputs a biological acquisition signal corresponding to the stimulation pattern, to the biological information acquisition unit 13. The biological information acquisition unit 13 accordingly acquires biological information of the user based on a biological information acquisition signal input from the stimulation control unit 22.

In Step S14, the stimulation control unit 22 primarily stores the biological information of the user that has been acquired by the biological information acquisition unit 13. In Step S15, the stimulation control unit 22 determines whether or not the number of times biological information of the user has been acquired is enough. In a case where the stimulation unit 12 applies a plurality of stimulation patterns to the user and the biological information acquisition unit 13 acquires biological information of the user that is obtained in response to the plurality of stimulation patterns, the stimulation control unit 22 determines whether or not biological information of the user has been acquired the number of times corresponding to the plurality of stimulation patterns (whether or not the number of times biological information of the user has been acquired is enough). Further, when the stimulation unit 12 applies one stimulation pattern to the user, and the biological information acquisition unit 13 calculates an average value by acquiring a plurality of pieces of biological information for the stimulation pattern, the stimulation control unit 22 determines whether or not biological information of the user has been acquired the number of times for calculating an average value (whether or not the number of times biological information of the user has been acquired is enough).

If the stimulation control unit 22 determines in Step S15 that the number of times biological information of the user has been acquired is not enough (No), the processing returns to Step S12, and the processing is repeated. On the other hand, if the stimulation control unit 22 determines that the number of times biological information of the user has been acquired is enough (Yes), the processing proceeds to Step S16. In Step S16, the personal information setting unit 23 sets, as a personal information determination value of the user, first personal information associating a plurality of stimulations applied to the user by the stimulation unit 12, and a plurality of pieces of biological information (a difference value of brain wave levels) acquired by the biological information acquisition unit 13 at this time. Then, in Step S17, the personal information storage unit 16 stores the personal information determination value set by the personal information setting unit 23, in association with an acquisition date, an ID of the user, or the like.

### Second Embodiment

### Authentication Apparatus

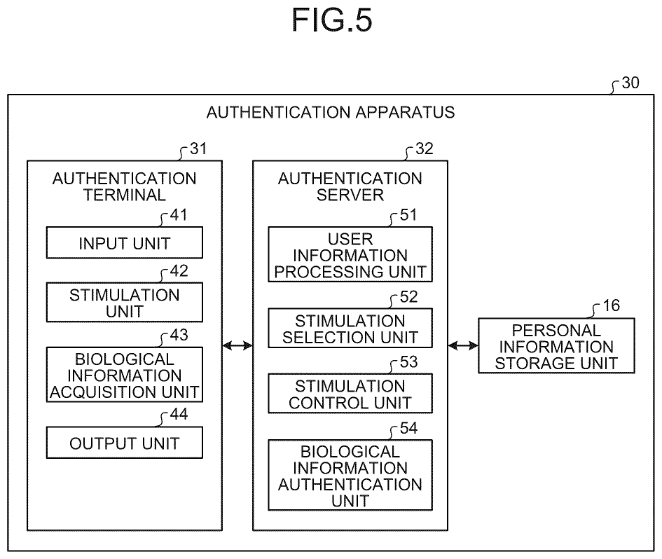

FIG. 5 is a block diagram illustrating an authentication apparatus according to a second embodiment.

As illustrated in FIG. 5, an authentication apparatus 30 includes an authentication terminal 31, an authentication server 32, and a personal information storage unit 16. The authentication terminal 31 is connected to the authentication server 32, and the personal information storage unit 16 is connected to the authentication server 32. Note that the authentication terminal 31 and the authentication server 32 are control devices, and include an arithmetic circuit such as a central processing unit (CPU), for example. Further, a storage unit (not illustrated) storing programs for performing various types of processing is connected to the authentication terminal 31 and the authentication server 32.

The authentication terminal 31 is a mobile terminal such as, for example, a personal computer, a smartphone, or a tablet, a card authentication terminal of a credit card or the like, or an authentication terminal or the like of an automated teller machine or the like.

The authentication terminal 31 includes an input unit 41, a stimulation unit 42, a biological information acquisition unit 43, and an output unit 44.

The input unit 41 is operable by the user, and can input various signals to the authentication server 32. The input unit 11 inputs, for example, a start signal for the user starting authentication processing of himself/herself, and an end signal for ending the authentication processing, to the authentication server 32. Using the input unit 11, the user inputs an ID of himself/herself or the like, for example, to the authentication server 32. The input unit 11 can be implemented by, for example, a touch panel, a button, a switch, a keyboard, or the like.

The stimulation unit 42 is connected to the authentication server 32. The stimulation unit 42 can apply a plurality of stimulations to the user. The authentication server 32 outputs a stimulation application signal to the stimulation unit 42 based on a program. The stimulation unit 42 applies a stimulation to the user based on the stimulation application signal input from the authentication server 32. The stimulation unit 42 applies a plurality of different stimulations to a predetermined number of sensory organs among five senses. The stimulation unit 42 applies a plurality of stimulations to the user at predetermined intervals. Note that the stimulation unit 42 has an almost similar configuration to that of the stimulation unit 12 of the first embodiment, and the detailed description will be omitted.

The biological information acquisition unit 43 is connected to the authentication server 32. The biological information acquisition unit 43 acquires biological information obtainable when the stimulation unit 42 applies a stimulation to the user. That is, if the authentication server 32 outputs a stimulation application signal to the stimulation unit 42 based on a program, the stimulation unit 42 applies a stimulation to the user. At this time, the authentication server 32 outputs a biological information acquisition signal to the biological information acquisition unit 43 based on a program. The biological information acquisition unit 43 accordingly acquires biological information of the user based on the biological information acquisition signal input from the authentication server 32. Note that the biological information acquisition unit 43 has an almost similar configuration to that of the biological information acquisition unit 43 of the first embodiment, and the detailed description will be omitted.

The output unit 44 can output processing results of the input unit 41, the stimulation unit 42, and the biological information acquisition unit 43 such as, for example, whether or not an ID of the user or the like has been input from the input unit 11, whether or not the stimulation unit 12 has applied a stimulation to the user, or whether or not the biological information acquisition unit 13 has acquired biological information of the user. The output unit 44 is, for example, a display device that displays an image, a video, and the like, or a sound output device that outputs sound.

The authentication server 32 is an information processing apparatus functioning as a server apparatus, and is a computer, a cloud system, or the like.

The authentication server 32 includes a user information processing unit 51, a stimulation selection unit 52, a stimulation control unit 53, and a biological information authentication unit 54.

The user information processing unit 51 performs processing of various types of information regarding the user. If an ID is input by the user using the input unit 11, the user information processing unit 51 reads a personal information determination value corresponding to the ID, from the personal information storage unit 16. If an ID and a start signal of authentication processing are input by the user using the input unit 11, the user information processing unit 51 outputs a start command signal of authentication processing to the stimulation selection unit 52 and the stimulation control unit 53.

The stimulation selection unit 52 selects a pattern by which the stimulation unit 12 applies a plurality of stimulations to the user. The stimulation selection unit 52 selects a predetermined stimulation pattern among a plurality of preset stimulation patterns. The stimulation pattern is a stimulation pattern generated by the stimulation generation unit 21 of the information processing apparatus 10 of the first embodiment, and stored in the personal information storage unit 16. The stimulation selection unit 52 sets a stimulation pattern stored as a personal information determination value that is read out by the user information processing unit 51 from the personal information storage unit 16. In a case where there is a plurality of stimulation patterns stored as a personal information determination value, the stimulation selection unit 52 selects one or a plurality of stimulation patterns.

The stimulation control unit 53 outputs a stimulation application signal of one stimulation pattern or a plurality of stimulation patterns selected by the stimulation selection unit 52, to the stimulation unit 42 based on a program. Further, simultaneously with outputting a stimulation application signal to the stimulation unit 42, the stimulation control unit 53 outputs a biological acquisition signal corresponding to the stimulation pattern, to the biological information acquisition unit 43 based on a program.

The stimulation unit 42 accordingly applies a stimulation to the user based on the stimulation application signal input from the stimulation control unit 53. Then, the biological information acquisition unit 43 acquires biological information of the user based on a biological information acquisition signal input from the stimulation control unit 53.

The biological information authentication unit 54 performs authentication of the user by comparing personal information associating a stimulation pattern applied to the user by the stimulation unit 12, and a difference value of brain wave levels of the user that has been acquired by the biological information acquisition unit 13, and a personal information determination value of the user that is stored in the personal information storage unit 16. That is, the user presets a personal information determination value of himself/herself using the information processing apparatus 10 (refer to FIG. 1), and stores the personal information determination value into the personal information storage unit 16. Thus, by comparing personal information set using the authentication terminal 31, and the personal information determination value stored in the personal information storage unit 16, it is possible to perform authentication of the user. In this case, comparison between personal information and a personal information determination value is preferably performed based on whether or not a difference between the personal information and the personal information determination value is equal to or smaller than a preset predetermined value.

The personal information storage unit 16 is connected to the authentication server 32. The personal information storage unit 16 is a part of components of the information processing apparatus 10 of the first embodiment.

Note that, in the above description, the user information processing unit 51, the stimulation selection unit 52, the stimulation control unit 53, and the biological information authentication unit 54 are provided in the authentication server 32, but these may be provided in the authentication terminal 31. In this case, the personal information storage unit 16 is in a cloud system, for example, and is connected to the authentication terminal 31.

Further, the information processing apparatus 10 of the first embodiment and the authentication apparatus 30 of the second embodiment have been described as different apparatuses, but these may be the same apparatus. In this case, for example, the input units 11 and 41, the stimulation units 12 and 42, the biological information acquisition units 13 and 43, the stimulation generation unit 21 and the stimulation selection unit 52, the stimulation control units 22 and 53, the output units 15 and 44, and the like can be shared.

### Authentication Method

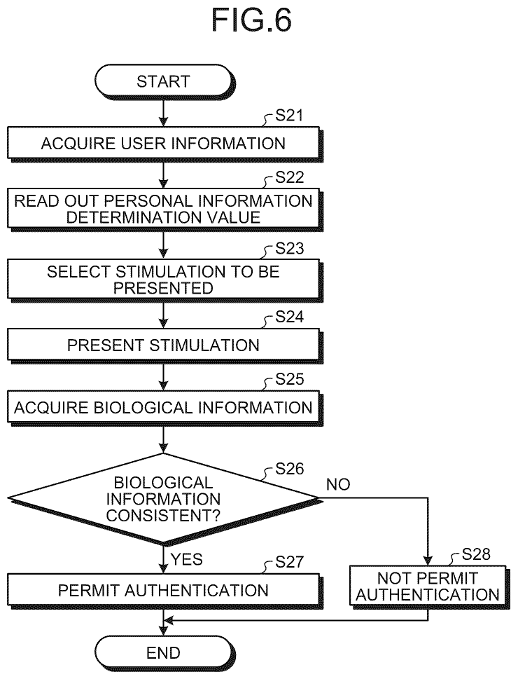

FIG. 6 is a flowchart illustrating an authentication method according to the second embodiment.

As illustrated in FIGS. 5 and 6, in Step S21, if a start signal of authentication processing and an ID are input from the input unit 41 of the authentication terminal 31, the user information processing unit 51 acquires various types of information regarding the user. In Step S22, the user information processing unit 51 reads out a personal information determination value corresponding to the acquired ID, from the personal information storage unit 16.

In Step S23, the stimulation selection unit 52 selects a pattern (stimulation pattern) by which the stimulation unit 42 applies a plurality of stimulations to the user. In this case, the stimulation selection unit 52 selects a stimulation pattern of a personal information determination value stored in the personal information storage unit 16. Note that, when the personal information determination value includes a plurality of stimulation patterns, the stimulation selection unit 52 selects one or a plurality of stimulation patterns from among the plurality of stimulation patterns. In Step S24, the stimulation control unit 53 outputs a stimulation application signal of the stimulation pattern selected by the stimulation selection unit 52, to the stimulation unit 42. The stimulation unit 42 accordingly applies a stimulation to the user based on the stimulation application signal input from the stimulation control unit 53.

In Step S25, simultaneously with outputting the stimulation application signal to the stimulation unit 42, the stimulation control unit 53 outputs a biological acquisition signal corresponding to the stimulation pattern, to the biological information acquisition unit 43. The biological information acquisition unit 43 accordingly acquires biological information of the user based on a biological information acquisition signal input from the stimulation control unit 53.

In Step S26, the biological information authentication unit 54 determines whether or not personal information associating a stimulation pattern applied by the stimulation unit 12, and a difference value of brain wave levels acquired by the biological information acquisition unit 13, and a personal information determination value of the user that is stored in the personal information storage unit 16 are consistent.

If the biological information authentication unit 54 determines in Step S26 that the personal information of the user that has been acquired by the authentication terminal 31 and the personal information determination value stored in the personal information storage unit 16 are consistent (Yes), in Step S27, the biological information authentication unit 54 permits authentication of the user. On the other hand, if the biological information authentication unit 54 determines that the personal information of the user that has been acquired by the authentication terminal 31 and the personal information determination value stored in the personal information storage unit 16 are not consistent (No), in Step S28, the biological information authentication unit 54 does not permit authentication of the user.

### Function Effect of Present Embodiment

An information processing apparatus of the present embodiment includes the stimulation unit 12 that applies a plurality of stimulations having the first relationship, the biological information acquisition unit 13 that acquires a brain wave level for the plurality of stimulations applied by the stimulation unit 12, and the personal information setting unit 23 that sets, as a personal information determination value, first personal information associating the plurality of stimulations having the first relationship that has been applied by the stimulation unit 12, and a difference value of a plurality of brain wave levels acquired by the biological information acquisition unit 13 at this time.

Thus, by setting, as the personal information determination value, the first personal information associating stimulations applied by the stimulation unit 12, and a difference value of brain wave levels acquired by the biological information acquisition unit 13, it is possible to easily acquire information unique to the user. In this case, because a brain wave level obtainable when a plurality of stimulations having the first relationship is applied is based on continuousness of memory of the user, an individual difference is often generated. Further, an individual difference among users is often generated also for a difference value of brain wave level, and it is possible to ensure high accuracy of personal identification.

In the information processing apparatus of the present embodiment, the stimulation unit 12 can apply a plurality of stimulations having the second relationship different from the first relationship, and the personal information setting unit 23 sets, as the personal information determination value, second personal information associating the plurality of stimulations having the second relationship that has been applied by the stimulation unit 12, and a difference value of a plurality of brain wave levels acquired by the biological information acquisition unit 13 at this time, together with the first personal information. Thus, it is possible to easily acquire information unique to the user, and set a personal information determination value with high accuracy of personal identification.

In the information processing apparatus of the present embodiment, the stimulation unit 12 can apply a plurality of stimulations in which an unrelated image is included in a plurality of stimulations having the first relationship, and the personal information setting unit 23 sets, as the personal information determination value, third personal information associating a plurality of stimulations in which in which an unrelated image is included in a plurality of stimulations having the first relationship, and a difference value of a plurality of brain wave levels acquired by the biological information acquisition unit 13 at this time, together with the first personal information. Thus, it is possible to easily acquire information unique to the user, and set a personal information determination value with high accuracy of personal identification.

An authentication apparatus of the present embodiment includes the information processing apparatus 10, the personal information storage unit 16 storing a personal information determination value set by the personal information setting unit 23, and the biological information authentication unit (authentication unit) 54 that performs authentication processing by comparing the first personal information associating a plurality of stimulations having the first relationship that has been applied by the stimulation unit 12, and a difference value of a plurality of brain wave levels acquired by the biological information acquisition unit 13 at this time, and a personal information determination value stored in the personal information storage unit 16. Thus, it is possible to acquire information unique to the user, and achieve improvement in accuracy of person authentication.

Heretofore, the information processing apparatus and the authentication apparatus according to the present disclosure have been described. Aside from the above-described embodiment, the present disclosure may be implemented in various different forms.

The components of the information processing apparatus and the authentication apparatus that are illustrated in the drawings indicate functional concepts, and physical configurations as illustrated the drawings are not always required. That is, a specific configuration of each apparatus is not limited to the configuration illustrated in the drawings, and all or part thereof may be functionally or physically dispersed or integrated in an arbitrary unit depending on a processing load, a usage situation, or the like of each apparatus.

The configurations of the information processing apparatus and the authentication apparatus are implemented by a program or the like that is loaded on a memory as software, for example. In the above-described embodiment, the configurations have been described as functional blocks to be implemented by cooperation between these hardware or software components. That is, these functional blocks can be implemented in various forms only by hardware, only by software, or by a combination of these.

The information processing apparatus and the program, and the authentication apparatus of the present disclosure can be applied to an authentication apparatus, for example.

According to the present disclosure, an effect that information unique to a user can be acquired is caused.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An information processing apparatus comprising:
a stimulation unit configured to apply a plurality of stimulations having first relationship;
a biological information acquisition unit configured to acquire a brain wave level for the plurality of stimulations applied by the stimulation unit; and
a personal information setting unit configured to set, as a personal information determination value, first personal information associating the plurality of stimulations having the first relationship that has been applied by the stimulation unit, and a difference value of a plurality of brain wave levels acquired by the biological information acquisition unit at this time.

2. The information processing apparatus according to claim 1,
wherein the stimulation unit can apply a plurality of stimulations having second relationship different from the first relationship, and
the personal information setting unit sets, as the personal information determination value, second personal information associating the plurality of stimulations having the second relationship that has been applied by the stimulation unit, and a difference value of a plurality of brain wave levels acquired by the biological information acquisition unit at this time, together with the first personal information.

3. The information processing apparatus according to claim 1,
wherein the stimulation unit can apply a plurality of stimulations in which an unrelated image is included in a plurality of stimulations having the first relationship, and
the personal information setting unit sets, as the personal information determination value, third personal information associating a plurality of stimulations in which in which an unrelated image is included in the plurality of stimulations having the first relationship, and a difference value of a plurality of brain wave levels acquired by the biological information acquisition unit at this time, together with the first personal information.

4. An authentication apparatus comprising:
the information processing apparatus according to any one of claim 1;
a personal information storage unit configured to store the personal information determination value set by the personal information setting unit; and
an authentication unit configured to perform authentication processing by comparing first personal information associating the plurality of stimulations having the first relationship that has been applied by the stimulation unit, and a difference value of a plurality of brain wave levels acquired by the biological information acquisition unit at this time, and the personal information determination value stored in the personal information storage unit.

5. An information processing method comprising:
a step of applying a plurality of stimulations having first relationship;
a step of acquiring a brain wave level for the plurality of applied stimulations; and
a step of setting, as a personal information determination value, first personal information associating the plurality of applied stimulations having the first relationship, and a difference value of a plurality of brain wave levels acquired at this time.

6. A non-transitory computer-readable storage medium storing a program for causing a computer operating as an information processing apparatus, to execute:
a step of applying a plurality of stimulations having first relationship;
a step of acquiring a brain wave level for the plurality of applied stimulations; and
a step of setting, as a personal information determination value, first personal information associating the plurality of applied stimulations having the first relationship, and a difference value of a plurality of brain wave levels acquired at this time.
